(19) United States
(12) Patent Application Publication
(10) Pub. No.: US 2012/0094384 A1
(43) Pub. Date: Apr. 19, 2012

(54) USE OF COMPOUNDS FOR DIFFERENTIATION OF CELLS

(76) Inventors: Donald Barton McPhail, Aberdeenshire (GB); Graeme James Cook, Aberdeenshire (GB); Andrew Scott Johnstone, Midlothian (GB)

(21) Appl. No.: 13/260,248

(22) PCT Filed: Apr. 9, 2010
(86) PCT No.: PCT/GB2010/000722

§ 371 Date: Dec. 16, 2011

(30) Foreign Application Priority Data
Apr. 9, 2009 (GB) 0906424.7

(51) Int. Cl. C12N 5/071; C12N 5/077; C12N 5/079

(52) U.S. Cl. 435/377

(57) ABSTRACT

The present invention provides a method for differentiating an undifferentiated cell, said method comprising contacting an undifferentiated cell with certain compounds of Formula (I) or a salt thereof.

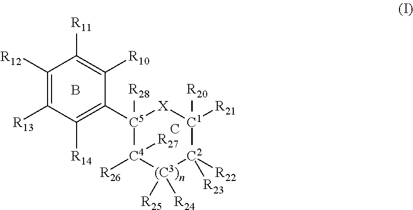

### FIELD OF THE INVENTION

The present invention relates to the differentiation of cells, and in particular the use of certain compounds for differentiating undifferentiated cells, for example stem cells.

### BACKGROUND TO THE INVENTION

Stem cells are unspecialised cells which are capable of differentiating into many different types of cells. Stem cell research is an area of great interest, with the potential to revolutionise treatment of numerous diseases such as Alzheimer's disease, Parkinson's disease, diabetes and heart failure. A number of different types of stem cells exist, including mesenchymal stem cells, haematopoietic stem cells, embryonic stem cells and induced pluripotent stem cells. Stem cells differ in their capacity to differentiate into particular cell types. Stem cells which are capable of differentiating into any type of cell in an organism, including embryonic cells, are termed totipotent. In humans, such totipotent stem cells are derived from the blastocyst and are termed embryonic stem cells. Stem cells which are capable of differentiating into any type of cell in an organism other than embryonic cells are termed pluripotent.

In order to treat such diseases using stem cells it is desirable to grow stem cells in the laboratory and induce them to differentiate into particular cell types as required. Stem cells can be induced to differentiate in vitro into different cell types using a number of agents, including retinoic acid, sodium butyrate and various growth factors. In this way, desired cell types can be produced by the use of particular differentiation-inducing agents.

EP-1834952-A1 discloses prenylflavanone compounds which are described as being useful for promoting the growth and development of neurons, the proliferation of neural stem cells and inducing the neural stem cells to differentiate into neurons.

EP-0743311-A1 discloses flavanonol derivatives which are described as accelerating differentiation and proliferation of hair matrix cells.

### SUMMARY OF THE INVENTION

The present inventors have identified certain compounds that are capable of causing the differentiation of undifferentiated cells such as stem cells.

In a first aspect, the present invention provides a method for differentiating an undifferentiated cell, said method comprising contacting an undifferentiated cell with a compound of Formula I or a salt thereof:

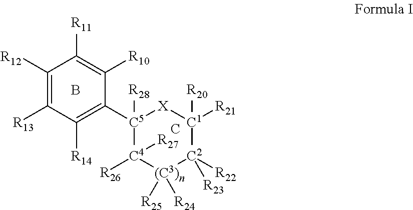

wherein:
A) R₁₂ and R₂₆ each independently represent —OH or a glycosidic functional group; R₁₀, R₁₁, R₁₃, and R₁₄ each independently represent H, —OH, nitro, halogen, amino, amido, cyano, carboxyl, sulphonyl, a glycosidic functional group, C₁₋₆ alkoxy-, hydroxy-C₁₋₆ alkyl-, C₁₋₆ alkoxy-C₁₋₆ alkyl-, or a saturated or unsaturated C₁₋₆ hydrocarbon chain which may be substituted with one or more of nitro, halogen, amino, amido, cyano, carboxyl, sulphonyl, hydroxyl, ketone or aldehyde groups; and wherein ring B comprises no more than one glycosidic functional group;
B) either a):
R₂₀ represents H or a C₂₋₃₀ saturated or unsaturated hydrocarbon chain;
R₂₁:
i) represents H;
ii) together with R₂₂ provides a second bond between C¹ and C²; or
iii) when X is —NR₁— and R₁ is not H or C₁₋₆ alkyl, together with R₁ provides a second bond between C¹ and N;
R₂₂:
i) represents H;
ii) together with R₂₃ forms ═O; or
iii) together with R₂₁ provides a second bond between C¹ and C²;
R₂₃:
i) represents H or a C₂₋₃₀ saturated or unsaturated hydrocarbon chain; or
ii) together with R₂₂ forms ═O;
   wherein at least one of R₂₀ and R₂₃ is a C₂₋₃₀ saturated or unsaturated hydrocarbon chain;
   or b):
R₂₀, R₂₁, R₂₂, and R₂₃ form part of a 5, 6 or 7 membered unsaturated ring including C¹ and C², which ring is substituted with at least one group which is a C₂₋₃₀ saturated or unsaturated hydrocarbon chain, which ring is optionally and independently further substituted with one or more groups selected from nitro, halogen, amino, amido, cyano, carboxyl, sulphonyl, hydroxyl, ketone, aldehyde and saturated or unsaturated C₁₋₆ hydrocarbon chain, which C₁₋₆ hydrocarbon chain may be substituted with one or more of nitro, halogen, amino, amido, cyano, carboxyl, sulphonyl, hydroxyl, ketone, aldehyde or nitron groups;
   C) n is 0 or 1, wherein when n is 0, either i) R₂₇ and R₂₈ represent H or ii) R₂₇ together with R₂₈ provide a second bond between C⁴ and C⁵; or when n is 1, either i) R₂₄ and R₂₅ together form ═O and R₂₇ and R₂₈ represent H or R₂₇ together with R₂₈ provide a second bond between C⁴ and C⁵, or ii) R₂₄ and R₂₅ represent H and R₂₇ and R₂₈ represent H or R₂₇ together with R₂₈ provide a second bond between C⁴ and C⁵ or iii) R₂₄ represents H, R₂₅ together with R₂₇ provide a second bond between C³ and C⁴, R₂₈ represents —OH and X is —O—;

D) X is —O—, —S— or —NR₁—, wherein R₁ i) represents H or C₁₋₆ alkyl, or ii) together with R₂₁ provides a second bond between C¹ and N;

wherein said C₂₋₃₀ saturated or unsaturated hydrocarbon chain of R₂₀, R₂₃ and the 5, 6 or 7 membered unsaturated ring is optionally and independently substituted with one or more groups selected from C₁₋₆ alkyl, C₁₋₆ alkoxy, hydroxy-C₁₋₆ alkyl, Cl, F, Br, I, —CN, —CO₂H, —CO₂C₁₋₆alkyl, —S(O)₂C₁₋₆alkyl, —S(O)₂-phenyl, —SC₁₋₆ alkyl, —NO₂, —OH, —CF₃, —N(R₂)(R₃), —NHC(O)NHC₁₋₆ alkyl, —C(O)N(R₂)(R₃), imine and substituted or unsubstituted triphenylphosphonium; and wherein one or more available —CH₂— groups present in the C₂₋₃₀ hydrocarbon chain of R₂₀, R₂₃ or the 5, 6 or 7 membered unsaturated ring is optionally and independently replaced by —O—, —C(O)—, —S(O)ₚ₋, or —N(R₂)—; wherein R₂ and R₃ each independently represent H or C₁₋₆ alkyl, and wherein p is 0 to 2;
and
wherein the total number of ═O on ring C is no greater than 1.

In some embodiments of the present invention, the compound of Formula I or salt thereof is a compound of Formula II or a salt thereof:

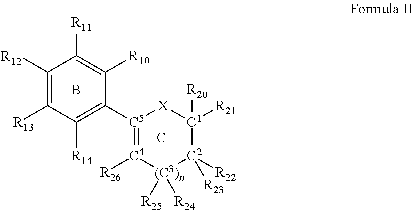

wherein:
A) R₁₂ and R₂₆ each independently represent —OH or a glycosidic functional group; R₁₀, R₁₁, R₁₃ and R₁₄ each independently represent H, —OH, nitro, halogen, amino, amido, cyano, carboxyl, sulphonyl, a glycosidic functional group, C₁₋₆ alkoxy-, hydroxy C₁₋₆ alkyl-, C₁₋₆ alkoxy-C₁₋₆ alkyl-, or a saturated or unsaturated C₁₋₆ hydrocarbon chain which may be substituted with one or more of nitro, halogen, amino, amido, cyano, carboxyl, sulphonyl, hydroxyl, ketone or aldehyde groups; and wherein ring B comprises no more than one glycosidic functional group;
B) either a):
R₂₀ represents H or a C₂₋₃₀ saturated or unsaturated hydrocarbon chain;
R₂₁:
i) represents H;
ii) together with R₂₂ provides a second bond between C¹ and C²; or
iii) when X is —NR₁— and R₁ is not H or C₁₋₆ alkyl, together with R₁ provides a second bond between C¹ and N;
R₂₂:
i) represents H;
ii) together with R₂₃ forms ═O; or
iii) together with R₂₁ provides a second bond between C¹ and C²;
   and
R₂₃:
i) represents H or a C₂₋₃₀ saturated or unsaturated hydrocarbon chain; or
ii) together with R₂₂ forms ═O;
   wherein at least one of R₂₀ and R₂₃ is a C₂₋₃₀ saturated or unsaturated hydrocarbon chain;
   or b)
R₂₀, R₂₁, R₂₂ and R₂₃ form part of a 5, 6 or 7 membered unsaturated ring including C¹ and C² (“A” ring), which ring is substituted with at least one group which is a C₂₋₃₀ saturated or unsaturated hydrocarbon chain, which ring is optionally and independently further substituted with one or more groups selected from nitro, halogen, amino, amido, cyano, carboxyl, sulphonyl, hydroxyl, ketone, aldehyde and saturated or unsaturated C₁₋₆ hydrocarbon chain, which C₁₋₆ hydrocarbon chain may be substituted with one or more of nitro, halogen, amino, amido, cyano, carboxyl, sulphonyl, hydroxyl, ketone, aldehyde or nitrone groups;
   C) n is 0 or 1, wherein when n is 1, either i) R₂₄ and R₂₅ together form ═O, or ii) R₂₄ and R₂₅ represent H;
   D) X is —O—, —S— or wherein R₁ represents i) H or C₁₋₆ alkyl, or ii) together with R₂₁ provides a second bond between C¹ and N;
   wherein said C₂₋₃₀ saturated or unsaturated hydrocarbon chain of R₂₀, R₂₃ or the 5, 6 or 7 membered unsaturated ring is optionally and independently substituted with one or more groups selected from C₁₋₆ alkyl, C₁₋₆ alkoxy, hydroxy-C₁₋₆ alkyl, Cl, F, Br, I, —CN, —CO₂H, —CO₂C₁₋₆ alkyl, —S(O)₂C₁₋₆ alkyl, —S(O)₂ phenyl, —SC₁₋₆ alkyl, —NO₂, OH, —CF₃, —N(R₂)(R₃), —NHC(O)NHC₁₋₆ alkyl, —C(O)N(R₂)(R₃), imine and substituted or unsubstituted triphenylphosphonium; wherein one or more available —CH₂— groups present in the C₂₋₃₀ hydrocarbon chain of R₂₀, R₂₃ or the 5, 6 or 7 membered unsaturated ring is optionally and independently replaced by —O—, —C(O)—, —S(O)ₚ₋, or —N(R₂)—;
   wherein R₂ and R₃ each independently represent H or C₁₋₆ alkyl, and wherein p is 0 to 2;
   and
   wherein the total number of ═O on ring C is no greater than 1.

In some or all embodiments of the present invention, the C₂₋₃₀ saturated or unsaturated hydrocarbon chain is a C₂₋₁₄ saturated or unsaturated hydrocarbon chain, typically a C₄₋₁₂ saturated or unsaturated hydrocarbon chain, more typically a C₆₋₁₂ saturated or unsaturated hydrocarbon chain, such as a C₈₋₁₀ or C₁₀₋₁₂ saturated or unsaturated hydrocarbon chain. Typically, the C₂₋₃₀ saturated or unsaturated hydrocarbon chain is C₂₋₃₀ saturated hydrocarbon chain, typically a C₂₋₁₄ saturated hydrocarbon chain, typically a C₄₋₁₂ saturated hydrocarbon chain, more typically a C₆₋₁₂ saturated hydrocarbon chain, such as a C₈₋₁₀ or C₁₀₋₁₂ saturated hydrocarbon chain, typically a C₁₀ or C₁₂ saturated hydrocarbon chain.

In some embodiments of the present invention, X is O.

In some embodiments of the present invention n=0. In other embodiments of the present invention

In some or all embodiments of the present invention, R₁₂ and R₂₆ may both represent OH; or one but not both of R₁₂ and R₂₆ may represent a glycosidic functional group, for example R₁₂ may be OH when R₂₆ is a glycosidic functional group or vice versa. In some embodiments of the present invention, one or both of R₁₁ and R₁₃ may represent OH; and/or R₁₀ and R₁₄ each independently represent H, OH or C₁₋₆-alkoxy-. An example of such a compound, wherein and R₂₇ together with R₂₈ provides a second bond between C⁴ and C⁵ is the compound of Formula III or a salt thereof:

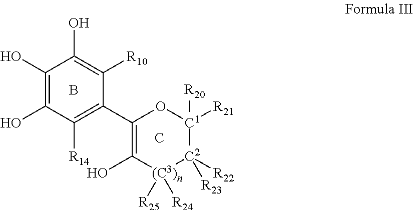

wherein:
A) R₁₀ and R₁₄ each independently represent H, —OH, nitro, halogen, amino, amido, cyano, carboxyl, sulphonyl, a glycosidic functional group, C₁₋₆ alkoxy-, hydroxy C₁₋₆ alkyl-, C₁₋₆ alkoxy-C₁₋₆ alkyl-, or a saturated or unsaturated C₁₋₆ hydrocarbon chain which may be substituted with one or more of nitro, halogen, amino, amido, cyano, carboxyl, sulphonyl, hydroxyl, ketone or aldehyde groups; and wherein ring B comprises no more than one glycosidic functional group;
B) either a):
R₂₀ represents H or a C₂₋₃₀ saturated or unsaturated hydrocarbon chain;
R₂₁:
i) represents H; or
ii) together with R₂₂ provides a second bond between C¹ and C²;
R₂₂:
i) represents H;
ii) together with R₂₃ forms ═O; or
iii) together with R₂₁ provides a second bond between C¹ and C²; and
R₂₃:
i) represents H or a C₂₋₃₀ saturated or unsaturated hydrocarbon chain; or
ii) together with R₂₂ forms ═O;
   wherein at least one of R₂₀ and R₂₃ is a C₂₋₃₀ saturated or unsaturated hydrocarbon chain;
   or b)
R₂₀, R₂₁, R₂₂ and R₂₃ form part of a 5, 6 or 7 membered unsaturated ring including C¹ and C² (“A” ring), which ring is substituted with at least one group which is a C₂₋₃₀ saturated or unsaturated hydrocarbon chain, which ring is optionally and independently further substituted with one or more groups selected from nitro, halogen, amino, amido, cyano, carboxyl, sulphonyl, hydroxyl, ketone, aldehyde and saturated or unsaturated C₁₋₆ hydrocarbon chain, which C₁₋₆ hydrocarbon chain may be substituted with one or more of nitro, halogen, amino, amido, cyano, carboxyl, sulphonyl, hydroxyl, ketone, aldehyde or nitrone groups;
   C) n is 0 or 1, wherein when n is 1, either i) R₂₄ and R₂₅ together form ═O, or ii) R₂₄ and R₂₅ represent H;
   wherein said C₂₋₃₀ saturated or unsaturated hydrocarbon chain of R₂₀, R₂₃ or the 5, 6 or 7 membered unsaturated ring is optionally and independently substituted with one or more groups selected from C₁₋₆ alkyl, C₁₋₆ alkoxy, hydroxy-C₁₋₆ alkyl, Cl, F, Br, I, —CN, —CO₂H, —CO₂C₁₋₆ alkyl, —S(O)₂C₁₋₆ alkyl, —S(O)₂ phenyl, —SC₁₋₆ alkyl, —NO₂, —OH, —CF₃, —N(R₂)(R₃), —NHC(O)NHC₁₋₆ alkyl, —C(O)N(R₂)(R₃), imine and substituted or unsubstituted triphenylphosphonium; wherein one or more available —CH₂— groups present in the C₂₋₃₀ hydrocarbon chain of R₂₀, R₂₃ or the 5, 6 or 7 membered unsaturated ring is optionally and independently replaced by —O—, —C(O)—, —S(O)ₚ₋, or —N(R₂)—;
   wherein R₂ and R₃ each independently represent H or C₁₋₆ alkyl, and wherein p is 0 to 2;
   and
   wherein the total number of ═O on ring C is no greater than 1.

In one embodiment of the invention, the compound is a compound of Formula III or a salt thereof, wherein:

R₁₀ and R₁₄ each represent H;
B) either a):
R₂₀ represents a C₂₋₁₄ saturated or unsaturated hydrocarbon chain; and
R₂₁ together with R₂₂ provides a second bond between C¹ and C²; and
R₂₃ represents H;
or b):
R₂₀, R₂₁, R₂₂ and R₂₃ form part of a 5, 6 or 7 membered unsaturated ring including C¹ and C² (“A” ring), which ring is substituted with at least one group which is a C₂₋₁₄ saturated or unsaturated hydrocarbon chain; and
   C) R₂₄ and R₂₅ together form ═O.

In this embodiment, the C₂₋₁₄ saturated or unsaturated hydrocarbon chain is typically a C₄₋₁₂ saturated or unsaturated hydrocarbon chain, more typically a C₆₋₁₂ saturated or unsaturated hydrocarbon chain, more typically a C₈₋₁₂ saturated or unsaturated hydrocarbon chain, typically a C₁₀₋₁₂ saturated hydrocarbon chain, more typically a C₁₀ or C₁₂ saturated hydrocarbon chain.

A further example of a compound of Formula II, but wherein X═O, n=1, R₂₄ together with R₂₅ forms ═O and R₂₇ together with R₂₈ provides a second bond between C⁴ and C⁵, is the compound of Formula VI or salt thereof:

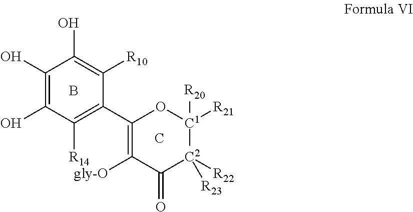

wherein:
A) R₁₀ and R₁₄ each independently represent H, —OH, nitro, halogen, amino, amido, cyano, carboxyl, sulphonyl, a glycosidic functional group, C₁₋₆ alkoxy-, hydroxy C₁₋₆ alkyl-, C₁₋₆ alkoxy-C₁₋₆ alkyl-, or a saturated or unsaturated C₁₋₆ hydrocarbon chain which may be substituted with one or more of nitro, halogen, amino, amido, cyano, carboxyl, sulphonyl, hydroxyl, ketone or aldehyde groups; and wherein ring B comprises no more than one glycosidic functional group; and O-gly represents a glycosidic functional group;
B) either a):
R₂₀ represents H or a C₂₋₃₀ saturated or unsaturated hydrocarbon chain;
R₂₁:
i) represents H; or
ii) together with R₂₂ provides a second bond between C¹ and C²;
R₂₂:
i) represents H; or
ii) together with R₂₁ provides a second bond between C¹ and C²; and
R₂₃ represents H, or a C₂₋₃₀ saturated or unsaturated hydrocarbon chain.
   wherein at least one of R₂₀ and R₂₃ is a C₂₋₃₀ saturated or unsaturated hydrocarbon chain;
   or b)
R₂₀, R₂₁, R₂₂ and R₂₃ form part of a 5, 6 or 7 membered unsaturated ring including C¹ and C², which ring is substituted with at least one group which is a C₂₋₃₀ saturated or unsaturated hydrocarbon chain, which ring is optionally and independently further substituted with one or more groups selected from nitro, halogen, amino, amido, cyano, carboxyl, sulphonyl, hydroxyl, ketone, aldehyde and saturated or unsaturated C₁₋₆ hydrocarbon chain, which C₁₋₆ hydrocarbon chain may be substituted with one or more of nitro, halogen, amino, amido, cyano, carboxyl, sulphonyl, hydroxyl, ketone, aldehyde or nitrone groups;
   wherein said C₂₋₃₀ saturated or unsaturated hydrocarbon chain of R₂₀, R₂₃ or the 5, 6 or 7 membered unsaturated ring is optionally and independently substituted with one or more groups selected from C₁₋₆ alkyl, C₁₋₆ alkoxy, hydroxy-C₁₋₆ alkyl, Cl, F, Br, I, —CN, —CO₂H, —CO₂C₁₋₆ alkyl, —S(O)₂C₁₋₆ alkyl, —S(O)₂ phenyl, —SC₁₋₆ alkyl, —NO₂, —OH, —CF₃, —N(R₂)(R₃), —NHC(C)NHC₁₋₆ alkyl, —C(O)N(R₂)(R₃), imine and substituted or unsubstituted triphenylphosphonium; wherein one or more available —CH₂— groups present in the C₂₋₃₀ hydrocarbon chain of R₂₀, R₂₃ or the 5, 6 or 7 membered unsaturated ring is optionally and independently replaced by —O—, C(O)—, —S(O)ₚ₋, or —N(R₂)—; and
   wherein R₂ and R₃ each independently represent H or C₁₋₆ alkyl, and wherein p is 0 to 2.

An example of a compound useful in the present invention where R₂₀, R₂₁, R₂₂ and R₂₃ form part of a 5, 6 or 7 membered ring is the compound of Formula VII or a salt thereof:

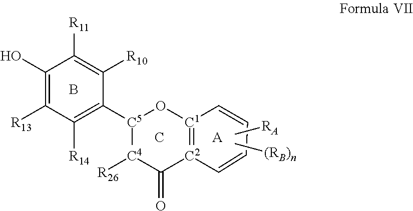

wherein
R_{A} is a C₂ to C₃₀ saturated or unsaturated hydrocarbon chain;
R₁₀, R₁₁, R₁₃, R₁₄ and R₂₆ each independently represent H, OH, a C₁₋₆ alkoxy, or a saturated or unsaturated C₁₋₆ hydrocarbon chain which may be substituted with one or more of nitro, halogen, amino, hydroxyl, ketone or aldehyde group;
optionally there is a double bond between C⁴ and C⁵ of the C ring; and
n represents 0 or 1; and
R_{B} is a C₂ to C₁₅ saturated or unsaturated hydrocarbon chain, where when R_{B} is present R_{A} and R_{B} are both C₂ to C₁₂ aliphatic alkyl chains.

The R_{A} group is preferably substituted on ring A at the para position with respect to C². The R_{A} group is preferably a C₆₋₁₅ saturated or unsaturated hydrocarbon chain.

Compounds of Formula VII are disclosed in WO 2004/007475.

In the present invention, the compound of Formula I or salt thereof may be an anthocyanin. Anthocyanins are generally known to exist in equilibrium between their hydrated hemiketal form and their flavylium cation form, both of which forms can be used in the present invention. Anthocyanins for use in the present invention are compounds of Formula I or salts thereof wherein:
R₁₂ represents OH
R₂₆ represents a glycosidic functional group R₂₅ together with R₂₇ provide a second bond between C³ and C⁴
R₂₈ represents OH
X═O
n=1
and R₂₀, R₂₁, R₂₂ and R₂₃ form part of a 6 membered unsaturated ring including C¹ and C², which ring is substituted with at least one group which is a C₂ to C₃₀ saturated or unsaturated hydrocarbon chain, which ring is optionally and independently further substituted with one or more groups selected from nitro, halogen, amino, amido, cyano, carboxyl, sulphonyl, hydroxyl, ketone, aldehyde and saturated or unsaturated C₁₋₆ hydrocarbon chain, which C₁₋₆ hydrocarbon chain may be substituted with one or more of nitro, halogen, amino, amido, cyano, carboxyl, sulphonyl, hydroxyl, ketone, aldehyde or nitrone groups.

Anthocyanins for use in the present invention can be represented by Formula VIIIHH, which is the structural formula of the compound in its hydrated hemiketal form, and Formula VIIIFC, which is the structural formula of the compound in its flavylium cation form. The flavylium cation form is also in equilibrium with the nonionic flavylium form represented by Formula VIIIFH:

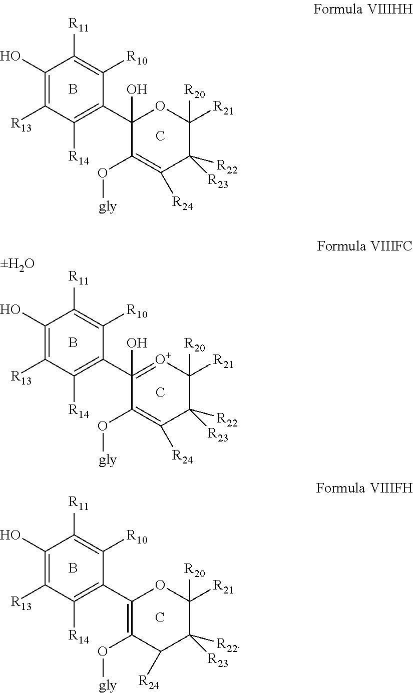

In each aspect of the present invention, when R₂₀, R₂₁, R₂₂ and R₂₃ in the compounds of Formula I or salts thereof form part of a 5, 6 or 7 membered unsaturated ring including C¹ and C², the ring is substituted with a C₂ to C₃₀ saturated or unsaturated hydrocarbon chain, as defined above, at either of the ortho, meta or para positions, typically at the meta position. The ring may be independently further substituted with one or more groups selected from nitro, halogen, amino, amido, cyano, carboxyl, sulphonyl, hydroxyl, ketone, aldehyde and saturated or unsaturated C₁₋₆ hydrocarbon chain, which C₁₋₆ hydrocarbon chain may be substituted with one or more of nitro, halogen, amino, amido, cyano, carboxyl, sulphonyl, hydroxyl, ketone, aldehyde or nitrone groups. In some embodiments, the ring is unsubstituted except for the C₂ to C₃₀ saturated or unsaturated hydrocarbon chain, as defined above.

By “substitution at the ortho position” is meant substitution on a carbon next to the C¹ on the ring. By “substitution at the meta position” is meant substitution on the carbon next to the ortho position remote from C¹. By “substitution at the para position” is meant substitution on the carbon next to the meta position remote from C¹. It will be appreciated by those skilled in the art that in the case of 5 membered rings, the para position may also be defined as the meta position.

In one embodiment, the compound of Formula I or salt thereof comprises a 5, 6 or 7 membered ring having the C₂ to C₃₀ hydrocarbon chain substituted at the meta or para position. For example, the compound of Formula I or salt thereof may comprise a 6 membered ring having the C₂ to C₃₀ hydrocarbon substituted at the meta or para position. Typically, the compound of Formula I or salt thereof comprises a 6 membered ring having a saturated C₂ to C₃₀ hydrocarbon, typically a saturated C₂ to C₁₄ hydrocarbon, more typically a saturated C₁₀ to C₁₂ hydrocarbon, substituted at the meta position.

The term “glycosidic functional group” is well known in the art, and is represented in the structural formulae herein as —O-gly. For avoidance of any doubt, however, a “glycosidic functional group” as used herein means a carbohydrate group linked to the main structure via a glycosidic bond. Preferably, the carbohydrate is a sugar. Preferably the sugar is glucose, rhamnose or rutinose.

In each embodiment of the present invention, the C₂₋₃₀ saturated or unsaturated hydrocarbon chain of R₂₀, R₂₃ and the 5, 6 or 7 membered unsaturated ring may have from two to twenty carbon atoms, preferably from six to fifteen carbon atoms. Suitably the hydrocarbon chain has a backbone having two, three, four, five, six, seven, eight, nine, ten, eleven, twelve, thirteen, fourteen, fifteen, sixteen, seventeen or eighteen consecutive carbon atoms.

The C₂₋₃₀ saturated or unsaturated hydrocarbon chain of R₂₀, R₂₃ and the 5, 6 or 7 membered unsaturated ring may include a —CH₂— group connecting to C¹, C² or the 5, 6 or 7 membered ring. This means, for example, that the C₂₋₃₀ hydrocarbon chain may not be an alkoxy group, though one or more carbon atoms within the C₂₋₃₀ hydrocarbon chain may be substituted with an alkoxy group.

The C₂₋₃₀ saturated or unsaturated hydrocarbon chain of R₂₀, R₂₃ and the 5, 6 or 7 membered unsaturated ring may be unsubstituted and is preferably saturated. The C₂₋₃₀ saturated or unsaturated hydrocarbon chain of R₂₀, R₂₃ and the 5, 6 or 7 membered unsaturated ring is preferably a straight hydrocarbon chain preferably comprising 6 to 15 carbon atoms.

When the C₂₋₃₀ saturated or unsaturated hydrocarbon chain is on a 5, 6 or 7 membered unsaturated ring, the ring is optionally and independently further substituted with one or more groups selected from nitro, halogen, amino, amido, cyano, carboxyl, sulphonyl, ketone, aldehyde and saturated or unsaturated C₂₋₁₅ hydrocarbon chain, which C₂₋₁₅ hydrocarbon chain may be substituted with one or more of nitro, halogen, amino, amido, cyano, carboxyl, sulphonyl, hydroxyl, ketone, aldehyde or nitrone groups. In one embodiment, the ring is unsubstituted except for the C₂₋₃₀ hydrocarbon chain. In another embodiment, the ring may be substituted with one or more groups selected from —NH2 and saturated or unsaturated C₂₋₁₅ hydrocarbon chain, which C₂₋₁₅ hydrocarbon chain may be substituted with one or more of nitro, halogen, amino, amido, cyano, carboxyl, sulphonyl, hydroxyl, ketone, aldehyde or nitrone groups.

Examples of specific compounds or salts thereof which are suitable for use in the present invention include:

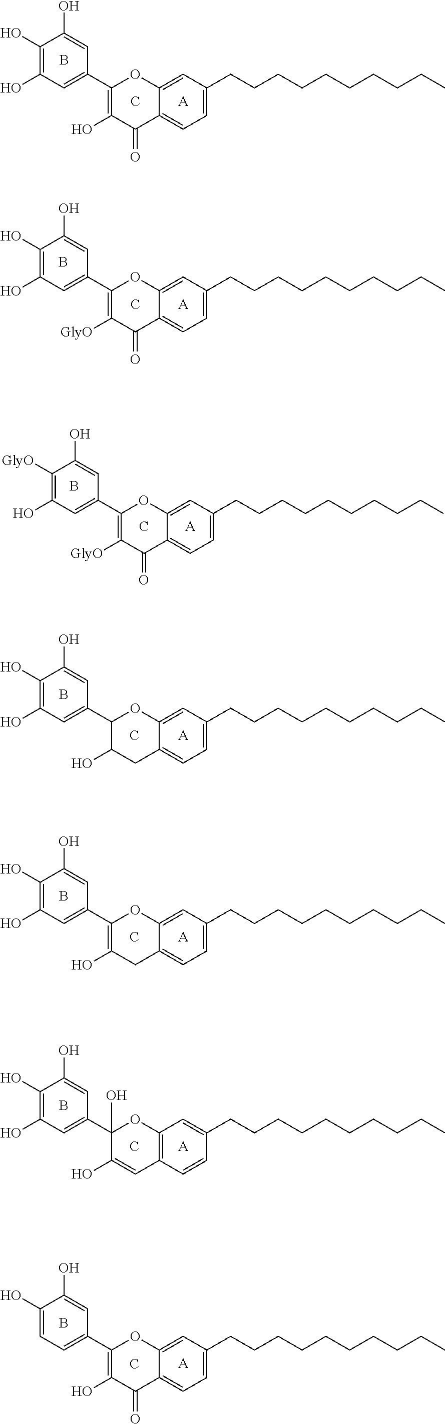

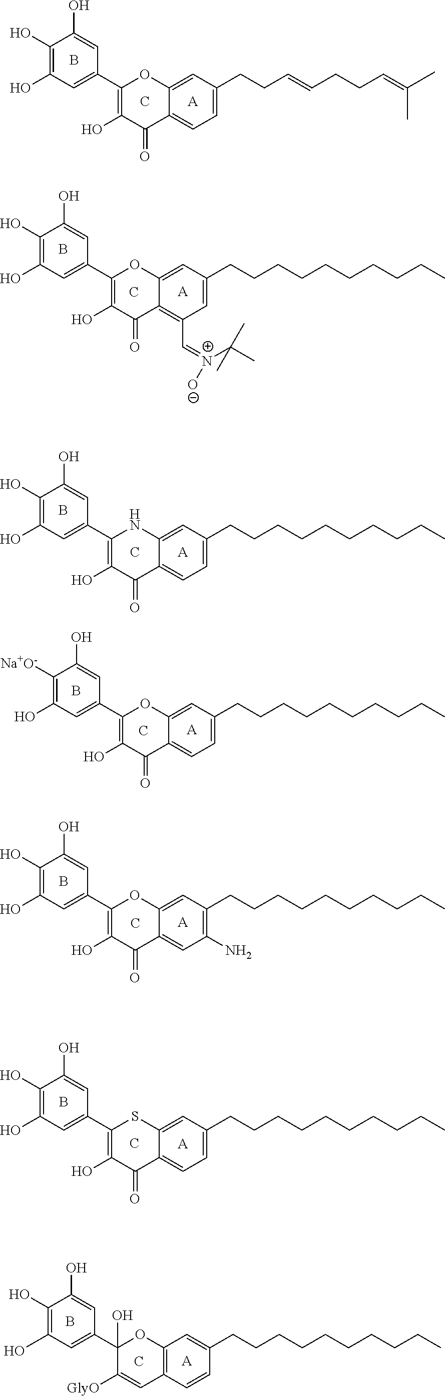

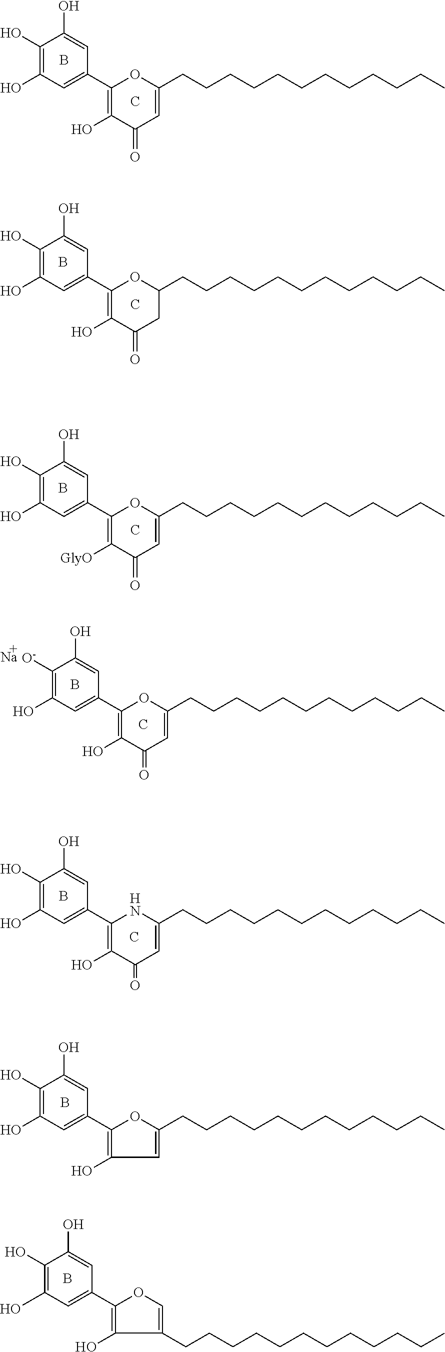

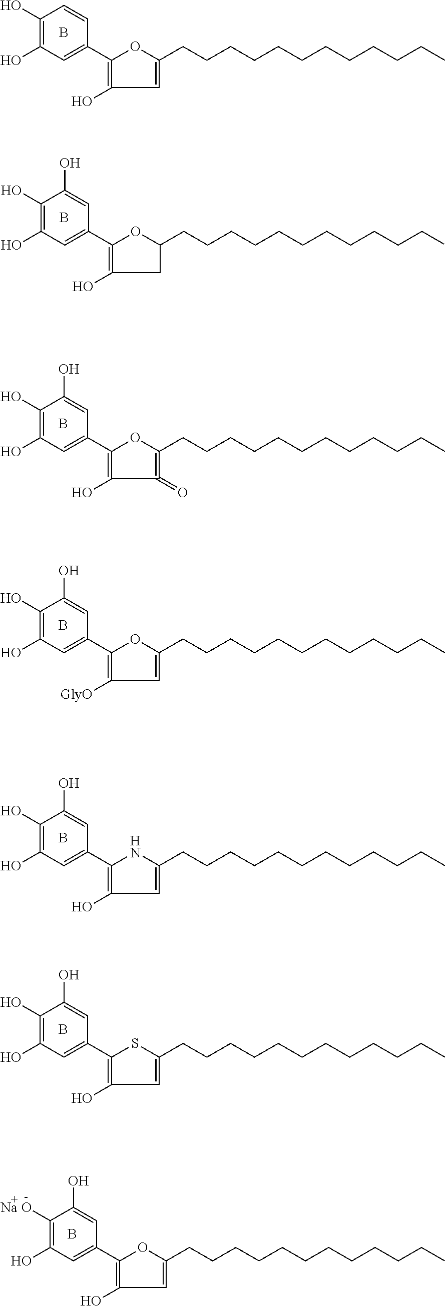

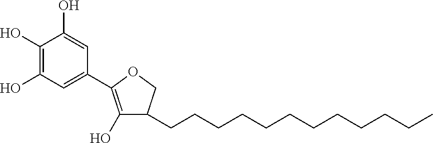

Preferred compounds which are suitable for use in the present invention are:

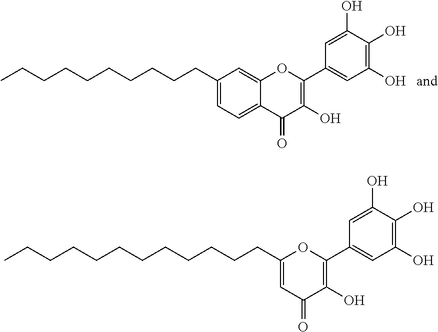

and salts of either thereof.

The methods of the present invention use compounds of the Formulae described herein or salts thereof. Typical examples of salts include hydrohalogenates (for instance, the hydrochloride, hydrobromide, or hydroiodide salt), inorganic acid salts (for instance, the sulphate, nitrate, perchlorate, phosphate, carbonate or bicarbonate salt), organic carboxylic acid salts (for instance, the acetate, maleate, tartrate, fumarate or citrate salt), organic sulfonic acid salts (for instance, the methanesulfonate (mesylate), ethanesulfonate, benzenesulfonate, toluenesulfonate or camphorsulfonate salt), amino acid salts (for instance, the aspartate or glutamate salt), quaternary ammonium salts, alkaline metal salts (for instance, the sodium or potassium salt) and alkaline earth metal salts (for instance, the magnesium or calcium salt).

The present invention relates to differentiating undifferentiated cells. Methods for differentiating an undifferentiated cell as claimed comprise contacting an undifferentiated cell with a compound of Formula I or a salt thereof. The methods of the present invention are typically carried out in vitro or ex vivo.

Typically, the undifferentiated cell is contacted with the compound of Formula I or a salt thereof by adding the compound or salt to the culture medium in which the undifferentiated cell is grown in vitro.

In the methods of the invention, the undifferentiated cell can be contacted with the compound of Formula I or a salt thereof as well as being contacted with one or more other reagents. Such reagents include, for example, growth factors including, but not restricted to, retinoic acid, BMP4, and activin A; and free radical generators and inducers of oxidative stress including, but not restricted to, tert-butyl hydroperoxide, menadione, buthionine sulphoximine (BSO) and dimethyl disulphide. Thus, in the methods of the invention, the undifferentiated cell can be contacted with the compound of Formula I or a salt thereof and with one or more growth factors and/or one or more free radical generators and inducers of oxidative stress. Alternatively or in addition to contacting the undifferentiated cell with one or more of these reagents, the undifferentiated cell can be subjected to one or more periods of oxygen deficit (hypoxia) or high oxygen concentrations (hyperoxia), i.e. subjected to modulation of oxygen levels.

The methods of the invention can comprise contacting the undifferentiated cell with one or more compounds of Formula I or salts thereof. Typically, the undifferentiated cell is contacted with a compound of Formula I or a salt thereof, or with a combination of 2, 3, 4, 5, 6 or more compounds of Formula I or salts thereof.

Undifferentiated cells which can be differentiated using the methods of the invention are typically stem cells. Stem cells are unspecialised cells which are capable of differentiating into various different types of cells and which are capable of self-renewal. Stem cells which can be differentiated using a method of the present invention include totipotent stem cells (capable of differentiating into embryonic and extraembryonic cell types), pluripotent stem cells (capable of differentiating into endoderm, mesoderm and ectoderm germ layers), and multipotent stem cells (capable of differentiating into a plurality of closely related cells). The methods of the present invention are therefore used to cause differentiation of completely undifferentiated cells.

Types of stem cells which can be differentiated using the methods of the present invention include embryonic stem (ES) cells (ESCs), adult stem cells and induced pluripotent stem (iPS) cells. Embryonic stem cells are derived from the blastocyst of a mammalian embryo and are totipotent. Embryonic stem cells were originally described by Evans and Kaufman (Nature, 292(5819): 154-156, 1981). Adult stem cells are pluripotent, and include hematopoietic stem cells and mesenchymal stem cells.

Stem cells which can be differentiated using the methods of present invention can be human or non-human. Typically, the stem cell is a mouse or human embryonic stem cell. Stem cells which can be differentiated using the methods of present invention include cancer stem cells and transgenic stem cells. Stem cells which can be differentiated using the methods of present invention include those produced from hybrid embryos or cytoplasmic hybrid (cybrid) embryos.

Embryonic stem cells can be isolated from blastocysts of members of the primate species (U.S. Pat. No. 5,843,780; Thomson et al., Proc. Natl. Acad. Sci. USA 92:7844, 1995). Human embryonic stem (hES) cells can be prepared from human blastocyst cells using primary mouse fibroblast feeder cells, according to the techniques described by Thomson et al. (U.S. Pat. No. 6,200,806; Science 282:1145, 1998; Curr. Top. Dev. Biol. 38:133, 1998) and Reubinoff et al., Nature Biotech. 18:399, 2000. hES cell lines can also be derived on human feeders (U.S. Pat. No. 6,642,048), or in conditions entirely free of feeder cells (US 2002/0081724) or Klimanskaya et al., Lancet, 365(9471):1636-41 (2005)). Equivalent cell types to hES cells include their pluripotent derivatives, such as primitive ectoderm-like (EPL) cells, as outlined in WO 01/51610. Embryonic stem cells may be chosen from embryonic stem cell lines or may be obtained directly from primary embryonic tissue.

It is not necessary for a human blastocyst to be disaggregated in order to produce the hES or embryonic stem cells for use in the method of the invention. hES cells can be obtained from established lines obtainable from public depositories, for example, the WiCell Research Institute (Madison Wis. USA), the American Type Culture Collection (Manassas Va., USA), the UK Stem Cell Bank (National Institute for Biological Standards and Control, UK) or the National Stem Cell Bank (University of Wisconsin-Madison, USA).

A number of embryonic stem cell lines have been established including, but not limited to, H1, H7, H9, H13 and H14 (Thompson et al.); hESBGN-01, hESBGN-02, hESBGN-03 (BresaGen, Inc., Athens, Ga.); HES-1, HES-2, HES-3, HES-4, HES-5, HES-6 (ES Cell International, Inc., Singapore); HSF-1, HSF-6 (University of California at San Francisco); I 3, I 4, I 6 (Technion-Israel Institute of Technology, Haifa, Israel); UCSF-1 and UCSF-2 (Genbacev et al., Fertil. Steril. 83(5):1517-29, 2005); lines HUES 1-17 (Cowan et al., NEJM 350(13):1353-56, 2004); and line ACT-14 (Klimanskaya et al., Lancet, 365(9471):1636-41, 2005).

Induced pluripotent cells are artificially derived from a non-pluripotent cell such as an adult somatic cell by the insertion of certain genes and are very similar to embryonic stem cells (Takahashi et al, Cell 131(5): 861-872, 2007; and Yu et al, Science 318(5858), 1917-1920, 2007). Stem cells are also found in the blood of the umbilical cord, and such umbilical cord blood stem cells can also be differentiated using the methods of the present invention.

US 2003/0113910 reports pluripotent stem cells derived without the use of embryos or fetal tissue. It may also be possible to reprogram other progenitor cells into hES cells by using a factor that induces the pluripotent phenotype (Chambers et al., Cell 113:643, 2003; Mitsui et al., Cell 113:631, 2003).

Other types of undifferentiated cells which can be differentiated using the methods of the present invention include embryonic germ (EG) cells and embryonic carcinoma (EC) cells.

Human embryonic germ (hEG) cells can be prepared from primordial germ cells as described in Shamblott et al., Proc. Natl. Acad. Sci. U.S.A. 95:13726, 1998 and U.S. Pat. No. 6,090,622.

The term “differentiating” means causing an undifferentiated cell to become differentiated, which in practice means that the undifferentiated cell loses its original capacity for differentiating into particular cell types and/or becomes committed to a particular cell lineage. In some embodiments, the method of the invention is used merely to cause the undifferentiated cell to move away from its original undifferentiated state. In these embodiments, the method of the invention can be used, for example, to cause a stem cell to no longer be totipotent, pluripotent or multipotent. The method of the present invention can be used not only to produce terminally differentiated cells, which have irreversibly differentiated into a particular cell type, but also to produce partially differentiated cells, which give rise to various different cell types. Such cells include the cells of the three embryonic germ cell layers: endoderm, mesoderm and ectoderm. In one embodiment of the invention, a method is provided for differentiating an undifferentiated cell into a partially differentiated cell. The present invention is therefore useful to produce cells which have not been committed to a particular pathway of differentiation. This embodiment of the invention finds use where cells are differentiated to a non-terminal state of differentiation before being transported or stored. The cells can then be induced to a terminally differentiated state when required, for example using a method of the invention.

The method of the present invention also extends to the production of embryoid bodies or embryonic bodies (EBs), which are multicellular aggregates of differentiated and undifferentiated cells and resemble early post-implantation embryos. In some embodiments, the present invention is used to cause EBs to move into a more differentiated state, for example to become committed to a particular cell lineage or to become partially differentiated or terminally differentiated, as defined herein.

Undifferentiated cells can be differentiated into various cell types using the method of the invention. For example, undifferentiated cells such as stem cells can be differentiated into, amongst other types of cells, epithelial cells, connective tissue cells, nerve cells such as neuronal cells, fat cells, pancreatic cells such as insulin-producing cells, liver cells, kidney cells, bone cells, hematopoietic cells, endothelial cells, retinal cells and smooth and striated muscle cells, including cardiomyocytes (heart muscle cells). Typically, undifferentiated cells such as stem cells are differentiated into neuronal cells or insulin-producing cells using the methods of the invention. In some embodiments, undifferentiated cells such as stem cells are partially differentiated using the methods of the invention, for example partially differentiated into a cell lineage that produces neuronal cells or insulin-producing cells.

As stated above, undifferentiated cells can also be differentiated into endoderm, mesoderm or ectoderm cells using a method of the invention.

Undifferentiated cells and differentiated cells can be identified, for example, by expression of particular marker genes. For example, stem cells can be identified by the expression of the marker genes Oct3/4 and Nanog. Differentiated cells can be identified, for example, by the expression of the following marker genes: Pax6, Sox1 and Zic1 for ectoderm; Nox1 for mesoderm; Brachyury (Bry; also symbolised by “T”) for mesendoderm; Sox17, CXCR4 and Foxa2 for definitive endoderm; and Sox7 and Afp for extra-embryonic endoderm. Other marker genes which can be used to identify differentiated cells include, for example, Fgf10, Neurod1, Podxl, Map4k1, Pak1, Fgf4 and Eomes.

Fgf10 (fibroblast growth factor) is implicated in numerous aspects of vertebrate embryonic development and adult tissue homeostasis. During development, it can act as an essential mediator of mesenchymal-epithelial interactions. It can regulate limb bud formation in the developing embryo. There is also some evidence that Fgf10 may affect development of the vertebrate lung. Brachyury gene, which is upregulated in mesendoderm, encodes for the transcription factor T. The T protein is essential for the differentiation and formation of posterior mesoderm and also for axial development in vertebrates. Neurod1 (neurogenic differentiation 1) is a class B basic helix-loop-helix (bHLH) transcription factor. It is expressed in pancreatic endocrine, intestine, and brain cells. It also activates the transcription of the insulin gene in pancreatic cells and is required for glucose homeostasis. It has a key role in the morphogenesis and differentiation of pancreatic β cells. Mutations are known to result in type II diabetes mellitus. Podxl (podocalyxin-like) encodes a member of the sialomucin protein family, an important component of podocytes, which are highly differentiated epithelial cells with interdigitating foot processes covering the outer aspect of the glomerular basement membrane. The encoded protein also plays a role in hematopoetic cell differentiation and is also expressed in vascular endothelium cells. Map4k1 (mitogen-activated protein kinase kinase kinase kinase 1) is thought to play a role in hematopoietic lineage decisions and growth regulation and also to have a role in relation to environmental stress response. It appears to act upstream of the JUN N-terminal. Pak1 (p21 (CDKN1A)-activated kinase 1) is a PAK protein. Such proteins are critical effectors that link RhoGTPases to cytoskeleton reorganization and nuclear signaling. These proteins serve as targets for the small GTP binding proteins Cdc42 and Rac. They have also been implicated in a wide range of biological activities. This gene regulates cell motility and morphology. The protein encoded by the Fgf4 (fibroblast growth factor 4) gene is a member of the fibroblast growth factor (FGF) family, which possess broad mitogenic and cell survival activities. They are involved in a variety of biological processes including embryonic development, cell growth, morphogenesis, tissue repair, tumor growth and invasion. This gene was identified by its oncogenic transforming activity. Studies have suggested a function in bone morphogenesis and limb development through the sonic hedgehog (SHH) signalling pathway. Eomes (eomesodermin homolog (Xenopus laevis)) is a gene shown to be essential during trophoblast differentiation and later, gastrulation. It has a likely role in brain development where it is required for specification and proliferation of intermediate progenitor cells and their progeny in the cerebral cortex.

Expression of such marker genes can be determined by any suitable method known in the art; for example qPCR.

The present invention therefore also encompasses a method of altering expression of a gene associated with differentiation in an undifferentiated cell, the method comprising contacting an undifferentiated cell with a compound of Formula I or a salt thereof as defined herein.

By “a gene associated with differentiation” is meant any gene whose protein product is involved in a differentiation pathway. Such genes therefore encode proteins that are involved in the process of a cell becoming partially or terminally differentiated. The expression of such genes may be upregulated or downregulated in this process, and thus “altering expression” includes both upregulating and downregulating expression. Upregulation and downregulation of expression relate respectively to an increase or decrease in expression, in comparison to the level of expression in a control population of cells. Such genes can also be described as differentiation marker genes, since their expression indicates differentiation of a cell into a particular cell type.

By “gene” is meant a nucleic acid encoding a protein, optionally together with its associated regulatory elements.

By “differentiation pathway” is meant a pathway of changes in gene expression, resulting in changes in the production of proteins, that causes a cell, such as an undifferentiated cell, to become partially or terminally differentiated. Such a pathway will typically involve a number of genes whose expression is inter-related. The expression of some genes may be upregulated in certain differentiation pathways whilst being downregulated in other differentiation pathways.

Genes whose expression can be altered using the method of the present invention include Pax6, Sox1, Zic1, Nox1, Bry, Sox17, CXCR4, Foxa2, Sox7 and Afp. As described above, these genes are associated with the following tissues: Pax6, Sox1 and Zic1 for ectoderm; Nox1 for mesoderm; Bry for mesendoderm; Sox17, CXCR4 and Foxa2 for definitive endoderm; and Sox7 and Afp for extra-embryonic endoderm.

Other genes whose expression can be altered using the method of the present invention include Fgf10, Neurod1, Podxl, Map4k1, Pak1, Fgf4, Eomes. These genes are associated with the tissues described above.

In this embodiment, the present invention therefore also encompasses a method of altering expression of a gene selected from the group consisting of Pax6, Sox1, Zic1, Nox1, Bry, Sox17, CXCR4, Foxa2, Sox7, Afp, Fgf10, Neurod1, Podxl, Map4k1, Pak1, Fgf4 and Eomes, the method comprising contacting an undifferentiated cell with a compound of Formula I or a salt thereof as defined herein. In one embodiment, the gene is Neurod1 and/or Fgf4.

The expression of any combination of these genes can be altered using the method of the present invention. For example, the method of the present invention can be used to alter the expression of any one of the genes, or any combination of 2, 3, 4, 5, 6, 7, 8, 9, 10, 11, 12, 13, 14, 15, 16 or all 17 of the genes.

The compounds described herein may induce the expression of certain genes associated with particular differentiation pathways, whilst inhibiting the expression of certain genes associated with other differentiation pathways, thus retarding the progress of other differentiation pathways.

The undifferentiated cells which can be differentiated using a method of the present invention are typically animal stem cells. The animal stem cells can be bird stem cells or fish stem cells. However, more typically, the stem cells are mammalian stem cells. Such mammalian stem cells include human and non-human stem cells. For example, non-human stem cells can be derived from rodents, such as a mice and rats; ungulates, such as cattle, sheep, goats and pigs; or other mammals such as cats, dogs, horses or rabbits.

In one embodiment, the present invention provides a method for differentiating an undifferentiated cell as claimed, said method comprising the following steps:

1. Embryonic stem cell (ESC) cultures are harvested by detaching the cells with trypsin. Once detached, trypsin inhibitor is added along with chemically-defined medium. Cells are then centrifuged, the supernatant removed, replaced with fresh chemically-defined medium and the cell pellet dispersed.
2. ESCs are then seeded at an appropriate number into an uncoated bacterial grade dish.
3. At this seeding stage, further chemically-defined medium is added to the dish in the presence of one or more compounds as defined herein. In addition, the experiment can be carried out in the absence of a compound as defined herein to allow comparison of the effects of the compounds on differentiation relative to the spontaneous differentiation that occurs with the native medium.
4. Optionally, the chemically-defined medium also contains growth factors including, but not restricted to, retinoic acid, BMP4, and activin A.
5. Optionally, the chemically-defined medium also includes free radical generators and inducers of oxidative stress including, but not restricted to, tert-butyl hydroperoxide (tBHP), menadione, buthionine sulphoximine (BSO) and dimethyl disulphide.
6. Optionally, the chemically-defined medium is also subjected to one or more periods of oxygen deficit.
7. The seeded cells are incubated at 37° C. with 5% CO₂ present and development of homogenous EBs encouraged by continual, gentle shaking of the dish for the first few days.
8. Cells are fed on a frequent basis (typically daily or every second day) by re-plating along with fresh chemically-defined medium, including any of the factors that has been added to the chemically-defined medium at the original plating.
9. EBs at the end of the required time period, or ESC's at initial plating on day 0 for comparison, are harvested, homogenised in Trizol and stored at −80° C. whilst awaiting analysis.
10. Analysis typically consists of: i) RNA isolation and measurement of the RNA concentration; ii) RT-PCR; and iii) qPCR with markers for required genes including, but not restricted to, GADPH, Nanog, Oct4, Pax6, Sox1, Zic1, Nox1, Bry, Sox17, CXCR4, Foxa2, Sox7 and Afp.
11. The resultant gene profiling is used to determine the differentiation of an undifferentiated cell.

This protocol can also be used for testing the effects of the compounds as defined herein on differentiation of ESCs to form EBs. In such a method, at the seeding stage 3, further chemically-defined medium is added to the dish in the presence or absence of one or more compounds as defined herein, to allow comparison of the effects of the compounds on differentiation relative to the spontaneous differentiation that occurs with the native medium. In step 11 of such methods, the gene profiling is used to determine the effects of one or more compounds as defined herein, with or without the other factors specified in steps 4, 5, and 6, on differentiation.

The invention will now be further described by way of reference to the following Examples and Figures which are provided for the purposes of illustration only and are not to be construed as limiting on the invention. Reference is made to the following Figures, in which:

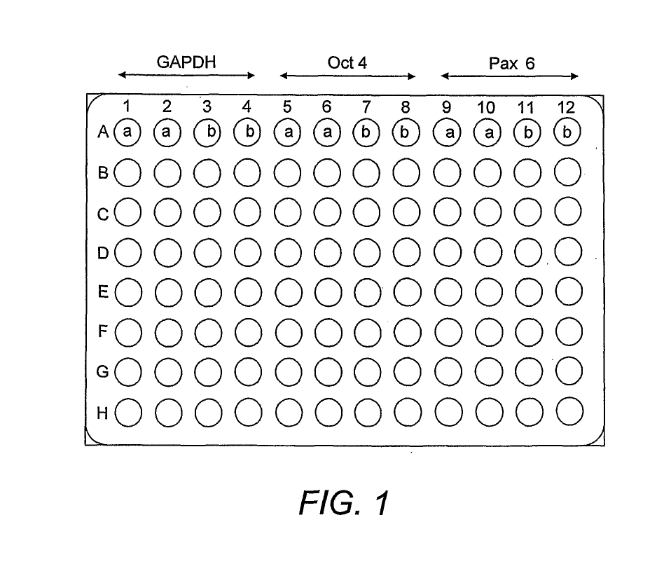

FIG. 1 shows a simplified plate layout for 2 cDNA samples (a and b) and 3 primers (GAPDH, Oct 4 and Pax6).

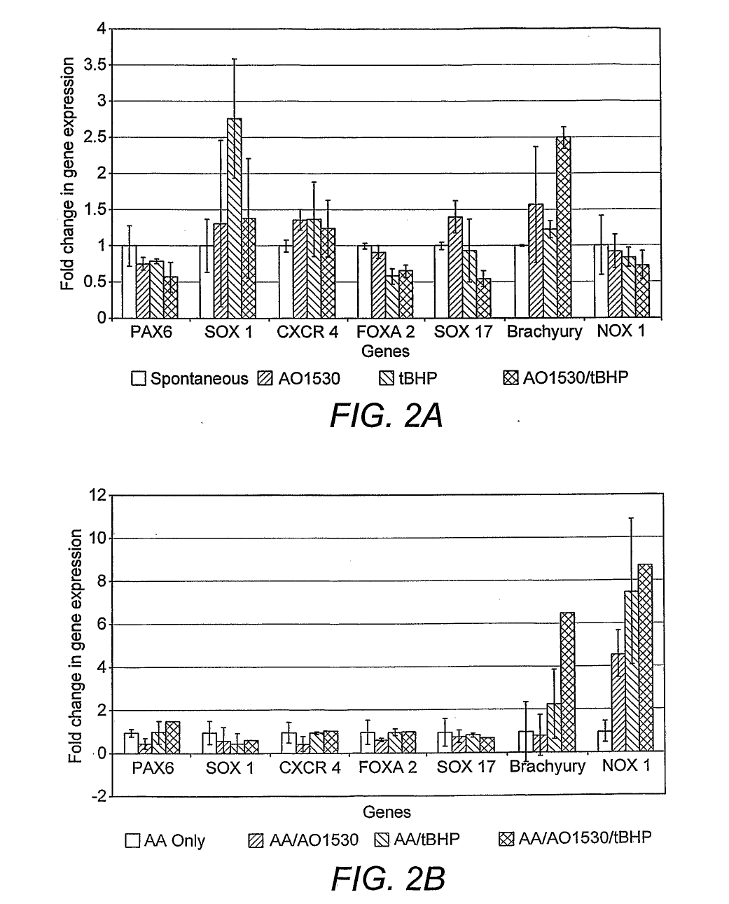

FIG. 2 shows (A) the effect of AO1530/tBHP on gene expression in spontaneously differentiating E14Tg2a cells; (B) the effect of AO1530/tBHP on gene expression in Activin A-treated E14Tg2a cells; (C) the effect of AO1530/tBHP on gene expression in BMP4-treated E14Tg2a cells; (D) the effect of AO1530/tBHP on gene expression in retinoic acid-treated E14Tg2a cells.

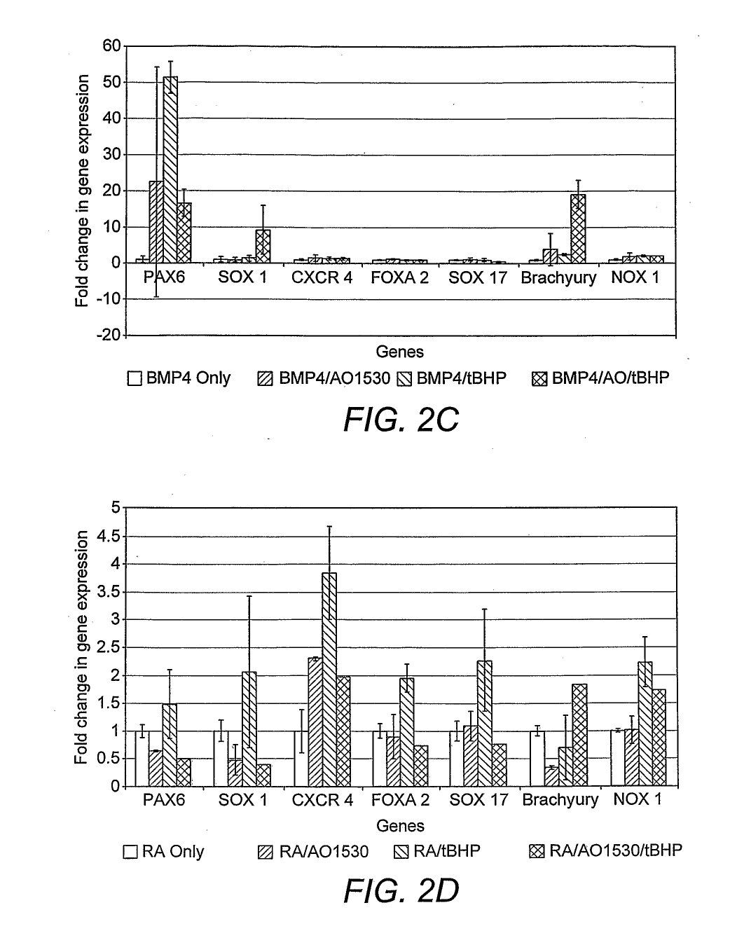

FIG. 3 shows the effect of AO-1-530 on the expression of differentiation-related genes relative to untreated (control) spontaneously differentiating mESC's. Raw data.

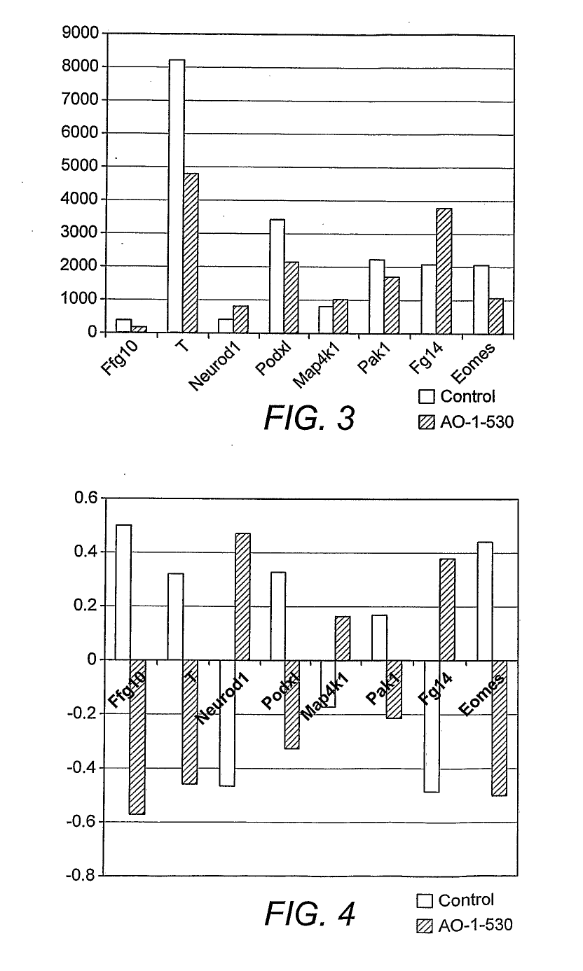

FIG. 4 shows the effect of AO-1-530 on the expression of differentiation-related genes relative to untreated (control) spontaneously differentiating mESC's. Normalised data.

### EXAMPLE 1

This Example describes the conditions and methods that were used to form embryonic bodies (EBs) and analyse subsequent mouse embryonic stem cell (MESC) differentiation.

### Preparation of Chemically Defined Medium (CDM)

When differentiating MESCs the cells must be grown in CDM. In CDM there are no unknown growth factors present which means that any change in the differentiation characteristics of the cells can be attributed to a known supplement.

CDM contains the following defined components (for 250 ml CDM):

| | Stock | | | Final | |
|---|---|---|---|---|---|
| Supplement | Concentration | Volume | | Concentration | |
| IMDM + GlutaMax | n/a | 117 | ml | n/a | |
| (Gibco) | | | | | |
| F12 + GlutaMax | n/a | 117 | ml | n/a | |
| (Gibco) | | | | | |
| BSA (Sigma: A9647) | 100 mg/ml | 12.5 | ml | 5 | mg/ml |
| Lipids | 100 x | 2.5 | ml | 1 x | |
| (Gibco: 11905-031) | | | | | |
| Transferrin | 30 mg/ml | 125 | μl | 15 | μg/ml |
| (Roche Diag: | | | | | |
| 10652202001) | | | | | |
| Insulin | 14 mg/ml | 125 | μl | 7 | μg/ml |
| (Sigma: 15500) | | | | | |
| Monothioglycerol | 98% | 9.75 | μl | 450 | μM |
| (Sigma: M-1753) | | | | | |

The BSA stock solution in PBS is prepared one day before it is needed, if possible. BSA dissolves well in PBS if it is kept at 4° C. overnight. The insulin solution is prepared only when needed. The insulin is dissolved in 1M acetic acid.

All supplements are pipetted into the top part of a steri-cup and the medium is then filtered into the sterile bottle by attaching the aspirator pump onto the nozzle on the filter. The bottle can be re-used with a new filter if kept sterile.

CDM should not be used after 7 days storage.

### Development of EBs

Uncoated 6 cm bacterial grade dishes were used to develop EBs. The MESCs were therefore in suspension throughout the period of differentiation; this aids the formation of EBs.

The procedure used for developing EBs was as follows:

The cells were harvested as per normal using CDM instead of supplemented Knockout Dulbecco's modified Eagle medium (KO DMEM). Briefly, KO DMEM was decanted from MESCs and cells washed with PBS. The PBS was decanted and the cells trypsinized. Once the cells had detached, trypsin inhibitor and then CDM was added to the detached cells. The cells were centrifuged then the supernatant aspirated and replaced with fresh CDM. The cell pellet was broken then the MESCs counted.

1.3×10⁶ cells were seeded in 4 ml of CDM per dish, ensuring that enough suspension had been prepared to plate all the dishes required. At this stage, the compound 7-decyl-3-hydroxy-2-(3,4,5-trihydroxy-phenyl)-chromen-4-one, referred to herein as AO1530 and having the structure set out below was added.

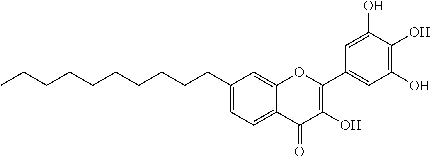

Additional reagents were also added to the cells as required at this stage at the necessary concentrations (for concentrations see Example 2). The additional reagents used in the experiments were the growth factors Activin A, Retinoic acid and BMP4; and the free radical generator and inducer of oxidative stress tert-butyl hydroperoxide (tBHP).

Each of the dishes was placed in an incubator at 37° C. (5% CO₂). The dishes were constantly and slowly shaken at the lowest speed possible (50/min) during incubation to aid the development of more homogenous EBs.

Day 0 samples were prepared for comparison with the end-point MESCs and processed as follows:

Two 500 μl aliquots of the original cell suspension were taken and placed in 1.5 ml sterile safe-lock eppendorf tubes. The cells were centrifuged at 7500 rpm (4° C.) for 4 minutes. The supernatant was decanted and 1 ml PBS added to the cells and the centrifugation described above repeated. The PBS was removed and the cells re-suspended in 1 ml Trizol by pipetting the sample a few times. The homogenised samples were incubated for 5 minutes at room temperature and then stored at −80° C.

### Feeding the EBs

The procedure that was followed when feeding the cells is laid out below:

The medium with the EBs in suspension was transferred to 15 ml falcon tubes. The EBs were allowed to settle to the bottom of the tubes (—10 minutes). The supernatant was aspirated with a pasteur pipette leaving 0.5 ml of the CDM in the tube and 3.5 ml of fresh CDM added to the cells. The required additional reagents (Activin A, retinoic acid and BMP4) were added to the CDM to obtain the required concentration. The EBs were plated into fresh 6 cm bacterial grade dishes and incubated while shaking at 37° C. (5% CO₂), and fed as appropriate every 24 or 48 hours.

### Harvesting the EBs

To harvest the EBs at the end of the experiment the following procedure was followed:

The medium with the EBs in suspension was transferred to 15 ml falcon tubes. The EBs were allowed to settle to the bottom of the tubes (10 minutes). The supernatant was aspirated with a pasteur pipette taking care not to disturb the EBs and 1 ml PBS added. The EBs were then transferred to 1.5 ml eppendorf tubes. The EBs were centrifuged and then the supernatant was aspirated, taking care not to disturb the cell pellet. The cells were then re-suspended in 1 ml Trizol by pipetting the sample a few times and stored at −80° C. until use.

### Trizol RNA Isolation

The suspensions were defrosted fully at room temperature and then 0.2 ml Chloroform per 1 ml Trizol suspension added. Each suspension was shaken vigorously by hand for 15 seconds and the suspensions incubated for 2 to 3 minutes at room temperature. The suspensions were then centrifuged at maximum speed for 15 minutes at 4° C. The upper aqueous phase was transferred to a fresh eppendorf tube taking care not to transfer or disturb the pink aqueous phase or the white debris in the middle phase. 0.5 ml Propan-2-ol was added to all tubes and each tube incubated for 10 minutes at room temperature. The mixture was centrifuged at maximum speed for 10 minutes at 4° C. After centrifugation a small white pellet was visible on the side of each tube. The supernatant was removed from each tube taking care not to disturb the pellet and 1 ml of 75% Ethanol (use sterile falcon tubes to prepare) added and mixed by hand. At this stage the suspension was either stored at −80° C. until further use or centrifuged at 7,500 G for 5 minutes at 4° C. If centrifuged, the ethanol was removed and the mixture left to air-dry for 5 to 10 minutes. The pellet was dissolved in DEPC water (30 μl if sufficient RNA had been obtained). The mixture was incubated for 10 minutes at 55 to 60° C. If required, the integrity of the RNA samples was determined by electrophoresis and analysis of the 18S and 28S rRNA bands.

### Measuring RNA Concentration

Before moving on to RT-PCR the concentration of the RNA samples was measured by following these steps:

68 μl TE buffer (pH 7.5) was aliquotted into sterile eppendorf tubes. 2 μl RNA solution (as described above) was added into the tubes and the absorbance of each RNA solution measured at 260, 270 and 280 nm using the entire 70 μl sample. A blank solution containing only TE buffer was run initially before any samples. Ratios of A260/A270 and A260/A280 were determined for each sample. Ideally, the A260/A270 ratio should be >1.1. Pure RNA has an A260/A280 ratio of 1.9-2.1. The concentration of each RNA sample was then determined by using the following calculation:

[RNA]_{μg/μl}=A260*Dilution Factor*40 μg/ml*1×10⁻³
Note: Dilution factor is normally 35

### Two-step RT-PCR

The following methods use the samples prepared in “Trizol RNA isolation” section above and are for preparing 1 batch of cDNA.

### DNA Digestion

0.5 ml eppendorf tubes were pre-chilled by placing them on ice. The following components were added to the pre-chilled eppendorf tubes in the order of:
a. 1 μg RNA
b. 1 μl 10× DNase 1 Buffer
c. 1 μl Amplification Grade DNase 1 (Invitrogen)
d. DEPC-H₂O to make the solution volume up to 10 μl

A ‘master-mix’ of 10× DNase 1 Buffer and DNase 1 was prepared to simplify sample preparation as the same volume of both went into each sample solution.

The solutions were incubated at room temperature for 15 minutes. The timer was started after adding and mixing the master-mix to the first sample, then master-mix was added to the other tubes. 1 μl of 25 mM EDTA was then added to the first sample and mixed thoroughly with a pipette tip. EDTA was then added to the remaining samples, in order for each of the samples to be incubated for exactly 15 minutes. All sample solutions were then heated for 10 minutes at 65° C. The solutions were then placed on ice for at least 2 minutes before micro-centrifuging for 1 minute.

### First Strand Synthesis

Whilst on ice, 1 μl of 5 ng/μl oligo dT was added to each sample and mixed thoroughly. All the sample solutions were heated for 10 minutes at 70° C. The samples were removed from the heat and placed on ice for at least 2 minutes before micro-centrifuging for 1 minute. The samples were placed on ice and then a master mix prepared for 1st strand synthesis by adding the following components in order (per sample):
i. 4 μl 5× Superscript Buffer
ii. 2 μl 0.1M DTT
iii. 1 μl RNasin
iv. 1 μl 10 mM dNTPs

8 μl of the master-mix was added to each sample and mixed thoroughly. 1 μA of (220 units/μl) superscript II reverse transcriptase was then added to each sample and mixed thoroughly, keeping the samples on ice. The samples were incubated for 50 minutes at 42° C. then the solutions placed at 70° C. for 15 minutes to inactivate the transcriptase enzyme. The solutions were then placed on ice to cool then micro-centrifuged for 1 minute.

At this point the samples were either:
a. stored at −80° C. until ready to continue with the analysis; or
b. made up to 50 μl by the addition of 29 μl DEPC-H₂O.
   qPCR Analysis

Before carrying out qPCR an appropriate collection of markers were selected to compare the sample cDNA to. These markers included the following genes:
Ectoderm: Pax6 and Sox1
Mesoderm: Nox1
Mesendoderm: Bry (Brachyury)
Definitive Endoderm: Sox17, CXCR4 and Foxa2

The following markers can also be used:
Internal control: GAPDH
Undifferentiated: Oct4 and Nanog
Ectoderm: Zic1
Extra-embryonic Endoderm: Sox7 and Afp

Analysis solutions were prepared using the following steps, all carried out while on ice:

0.5 ml eppendorf tubes are pre-chilled by placing them on ice. A master-mix of each selected primer is prepared by adding the following components to each tube (per sample. Samples were analysed in duplicate, e.g. for 8 samples 16× the following volumes were used):
a. 10 μl Sensimix
b. 4 μl DEPC-H₂O
c. 1 μl primer of interest

15 μl of each primer master-mix solution was aliquotted, as appropriate, into the wells of a 96-well plate that can be used for qPCR. 50 of sample cDNA was added to each aliquot of primer solution as appropriate. A simplified plate layout is shown in FIG. 1 for 2 cDNA samples (a and b) and 3 primers (GAPDH, Oct 4 and Pax6).

The plate was centrifuged at 1000 g for 1 minute then the plate analysed using a PCR plate reader. The results were quantified by using delta Ct (ΔCt) where Ct is the cycle threshold; the point when the fluorescence reading surpasses a set baseline. ΔCt was calculated as follows: ΔCt=Ct_{gene of interest} Ct_{GADPH}

The ΔCt values were then used in the following calculation to determine whether there were any changes in gene expression between the different treatment groups: 2^{-Δct}

### EXAMPLE 2

### Analysis of Spontaneously Differentiating EBs and Retinoic Acid, Activin A (AA) and BMP4 treated EBs co-incubated with AO1530 and tert-butylhydroperoxide (tBHP)

This Example describes an experiment that was undertaken to analyse the treated EBs that were harvested as described in Example 1. The undertaken analysis includes RNA isolation, two step RT-PCT and qPCR.

### Experimental Conditions

Differentiation of the E14Tg2a cells (mouse embryonic stem cells) under the following conditions was analysed (all conditions carried out in duplicate):
1. Spontaneously differentiating EBs
2. Spontaneously differentiating EBs/0.5 μM AO1530
3. Spontaneously differentiating EBs/3 μM tBHP
4. Spontaneously differentiating EBs/0.5 μM AO1530/3 μM tBHP
5. 1 μM RA-treated EBs
6. 1 μM RA-treated EBs/0.5 μM AO1530
7. 1 μM RA-treated EBs/3 μM tBHP
8. 1 μM RA-treated. EBs/0.5 μM AO1530/3 μM tBHP
9. 100 ng/ml AA-treated EBs
10. 100 ng/ml AA-treated EBs/0.5 μM AO1530
11. 100 ng/ml AA-treated EBs/3 μM tBHP
12. 100 ng/ml AA-treated EBs/0.5 μM AO1530/3 tBHP
13. 20 ng/ml BMP4-treated EBs
14. 20 ng/ml BMP4-treated EBs/0.5 μM AO1530
15. 20 ng/ml BMP4-treated EBs/3 μM tBHP
16. 20 ng/ml BMP4-treated EBs/0.504 AO1530/3 μM tBHP

In summary, the experiments were carried out on either spontaneously differentiating EBs, Activin A-treated, BMP4-treated or Retinoic acid-treated EBs that have been co-incubated with either 0.5 μM AO1530, 3 μM tBHP or a combination of both.

### Method

The method used in these experiments was as described in Example 1. Briefly:
1. Treated EBs were removed from −80° C. and fully defrosted
2. RNA was extracted from the cells by the Trizol RNA isolation method.
3. The concentration of RNA solutions was determined by measuring absorbance at A260 and A280 nm and using the following calculation:

[RNA]_{μg/μl}=A260*Dilution Factor*40 μg/ml*1×10⁻³
4. RNA solutions were prepared for two-step RT-PCR by carrying out DNA digestion then first strand synthesis.
5. Appropriate markers of cellular fate were chosen to analyse the DNA samples against (Pax 6, Sox 1, Cxcr 4, Foxa 2, Sox 17, Brachyury and Nox 1)
6. qPCR analysis was undertaken on each sample of DNA
7. Cell differentiation was analysed by comparing delta Ct values of each batch of DNA, where delta Ct was calculated as follows:

ΔCt=Ct_{gene of interest}−Ct_{GAPDH}
8. The ΔCt values were then used in the following calculation to determine whether there were any changes in gene expression between the different treatment groups: 2^{-Δct}

### Results

The results are shown in FIG. 2 and summarised in Table 1, in which:
↓—down-regulation of gene expression
↑—up to 10 fold up-regulation of gene expression
↑↑—between 10-20 fold up-regulation of gene expression
↑↑↑—over 20 fold up-regulation of gene expression
NSC—No significant change

These results illustrate that each treatment (AO1530, tBHP or AO1530/tBHP) has at least some effect on gene expression in the various treatments that were used throughout this experiment.

### Spontaneously Differentiating (SD) EBs

The largest change in gene expression occurred for Sox 1 (↑˜2.8 fold) when the EBs were treated with tBHP. This indicates that when the EBs, are spontaneously differentiating oxidative stress may play a role in the up-regulation of Sox 1. When the EBs were treated with AO1530 or AO1530/tBHP this increase in Sox 1 expression was almost reduced to the base level, suggesting that oxidative stress plays a significant role in Sox 1 expression in spontaneously differentiating EBs and that AO-1-530 can strongly inhibit this amplification. For the definitive endoderm markers Cxcr 4 and Sox 17 AO1530 increased expression by ˜1.4 fold in both cases, while Foxa 2 expression was decreased by tBHP and AO1530/tBHP treatment and Sox 17 expression was decreased by AO1530/tBHP treatment. These results suggest that the expression of these definitive endoderm markers may be effected by regulation of oxidative stress. Finally, the expression of the mesoendoderm marker brachyury was increased ˜2.5 fold by treatment with AO1530/tBHP. This is interesting as when the EBs were treated with only AO1530 or tBHP no apparent change in gene expression occurred. As AO1530 is an antioxidant and tBHP is an oxidant, in theory it would be expected that their actions should cancel each other out and not result in a synergistic increase in gene expression when each compound alone does not affect gene expression. AO1530, when oxidised by quenching free radicals (eg generated by tBHP), will form a quinone and it seems that it is this structure that is ultimately affecting the expression of brachyury in this case.

### Activin A-treated EBs

Following treatment of the EBs no significant changes was observed in any of the genes except for the brachyury and the mesoderm marker Nox 1. Brachyury is increased significantly (˜6.5 fold) by treatment with AO1530/tBHP combination. This mimics the effect observed in brachyury expression in spontaneously differentiating treated with AO1530/tBHP. Nox 1 expression was significantly increased by AO1530 (˜4.6 fold), tBHP (˜7.5 fold) and AO1530/tBHP (8.7 fold). The error bar for tBHP is quite large, however even when taking this into account it appears that tBHP would still have a substantial effect on Nox 1 expression. It seems as though in this case AO1530 and tBHP act synergistically to increase gene expression of Nox 1.

### BMP4-Treated EBs

In this set of experiments, AO1530 treatment does not affect the expression of any of the genes analysed. However, treatment of the EBs with tBHP or AO1530/tBHP does lead to some interesting data. The expression of Pax 6 is very significantly amplified following treatment with tBHP (˜51.5 fold). However, addition of AO1530 to the peroxide-challenged cells substantially reduces this amplification to ˜16.7 fold. This indicates that oxidative stress is an important factor in the up-regulation of Pax 6 expression and that AO1530 can effectively inhibit this process. Once again it appears that AO1530 and tBHP act synergistically to affect the expression of various genes that alone they do not necessarily have an effect on. Sox 1 and Brachyury expression is increased by ˜9.3 fold and 19.1 fold respectively while Sox 17 expression is decreased to ˜0.4 fold of the control. tBHP treatment does increase the expression of Brachyury (˜2.6 fold) however this increase is dwarfed by the expression observed in AO1530/tBHP treated EBs. Again this suggests an amplification role for the tBHP-oxidised form of AO1530. As Brachyury is a marker for mesoendoderm, then the tBHP/AO1530 combination may provide a potent method to form the mesoendoderm. Nox 1 expression is similarly increased in both tBHP (2 fold) and AO1530/tBHP (˜1.9 fold) treated EBs indicating that it is the tBHP treatment that is the major factor in the regulation of this gene and perhaps not via an oxidative mechanism as AO1530, in this instance, is not inhibitory.

### Retinoic Acid-Treated EBs

For the ectoderm markers Pax 6 and Sox 1 treatment with either AO1530 or AO1530/tBHP resulted in a decrease in gene expression while treatment with tBHP increased gene expression in both. These results illustrate that oxidative stress may be a factor in the expression of both Pax 6 and Sox 1 and treatment with antioxidants may result in a significant down-regulation of these genes in retinoic acid-treated EBs. For the definitive endoderm markers, Cxcr 4, Foxa 2 and Sox 17 treatment with tBHP resulted in an increase of all three genes (˜3.8, ˜2 and ˜2.3 fold respectively). This increase in expression for Cxcr 4, Foxa 2 and Sox 17 was ameliorated in AO1530/tBHP treated EBs (˜2, ˜0.7 and ˜0.8 fold changes respectively) and was not observed in AO1530 treated EBs for Foxa 2 and Sox 17 expression although Cxcr 4 expression was slightly increased (2.3 fold). This implies that oxidative stress is involved in the up-regulation of the definitive endoderm markers, Cxcr 4, Foxa 2 and Sox 17 and that the quenching of oxidative stress through treatment with antioxidants negates this up-regulation of gene expression. One of the most interesting aspects of this experiment is the apparently conflicting effects of AO1530 and AO1530/tBHP treatment on brachyury expression. When the EBs are treated with only the antioxidant AO1530, brachyury expression decreases significantly (˜0.3 fold change). However, as is seen in each of the different experimental sets described above, when the EBs are co-treated with both AO1530 and tBHP brachyury expression increases significantly (˜1.8 fold). This again suggests that the synergism between AO1530 and tBHP is important in the regulation of brachyury expression. In the case of Nox 1, gene expression is up-regulated in both tBHP and AO1530/tBHP treatment although it is in the tBHP treated EBs that Nox 1 expression has increased the most (˜2.2 fold compared to ˜1.7 fold following AO1530/tBHP treatment). This type of increased Nox 1 expression has been observed in each of the different conditions used except for in spontaneously differentiating EBs. This suggests that oxidative stress plays an important role in the up-regulation of Nox 1.

### SUMMARY

These results demonstrate that:
i) In BMP4-treated EBs Pax 6 expression is vastly amplified following tBHP treatment and this up-regulation can be ameliorated substantially if tBHP is co-incubated with AO1530. This effect is also observed in the expression of the definitive endoderm markers Cxcr 4, Foxa 2 and Sox17 in retinoic acid-treated EBs and possibly Sox 1 in spontaneously differentiating EBs.
ii) Brachyury expression is up-regulated in each of the different conditions following AO -1530/tBHP treatment. An oxidised form of AO1530 may be responsible for this as neither AO1530 or tBHP treatment on its own affects gene regulation as significantly as when used together.
iii) Nox 1 expression is up-regulated in Activin A, BMP4 and Retinoic acid-treated EBs following tBHP and AO1530/tBHP treatment. The levels of up-regulation are very similar following both treatments indicating that tBHP treatment is the major factor in the expression of Nox 1.

### EXAMPLE 3

### Effects of Compound AO-1-530 on Mouse Embryonic Stem Cell Differentiation as Assessed by GeneChip Array

### Experimental

Undifferentiated mouse embryonic stem cells (mESC's) were seeded at a density of 1.3×10⁶ cells on 6 cm uncoated bacterial grade dishes. Spontaneous differentiation was initiated by removal of LIF from the new incubation medium, which consisted of 2% serum KO DMEM+/−AO-1-530 (0.5 μM). AO-1-530 is the same compound used in Examples 1 and 2. There were three treated and three control replicates.

Cells were incubated on a shaking platform at 37° C. for 24 hours at which time the plating media was removed and fresh medium, or AO-1-530 treated medium added as appropriate. This was repeated at 48 hours and 72 hours.

The partially-differentiated cells or embryoid bodies (EB's) were harvested at 96 hours, washed with PBS, re-suspended in Trizol and stored at −80° C. awaiting RNA extraction. The cells were subsequently thawed and RNA extracted using a standard Trizol isolation procedure with an additional clean-up performed using a Qiagen RNeasy Mini kit and DNase digestion as per the manufacturer's instructions. cRNA was prepared from the tRNA, then hybridised to the Affymetrix GeneChip Mouse Exon 1.0ST array, washed, stained then scanned.

### Results and Discussion

Statistical evaluation on the control and treated samples was as follows:

### Test: T-test

P-value cut-off: 0.05
P-value computation: Asymptotic
Multiple testing correction: Benjamini-Hochberg

Treatment of the spontaneously differentiating mouse embryonic stem cells with AO-1-530 significantly altered (P<0.05) expression of 315 genes compared with the controls. Of this gene set, 8 genes known to be involved with stem cell differentiation processes were identified: Fgf10, Bry, Neurod1, Podxl, Map4k1, Pak1, Fgf4, Eomes. This subgroup of the 315 gene dataset is shown in Table 2, along with the corrected P-values, raw data, normalized data, gene codes and descriptions. The raw and normalized data is shown in FIGS. 3 and 4.

The data indicates that treatment with AO-1-530 results in an increase in Neurod1 and Fgf4 genes relative to the control cells with the remaining 6 genes being decreased relative to the control cells.

These results demonstrate that AO-1-530, a representative antioxidant of the category described herein, influences stem cell differentiation as evidenced by its effects on genes associated with this process.

TABLE 1

| | Gene Expression Relative to Control | | | | | | |
|---|---|---|---|---|---|---|---|
| Treat- | Pax | Sox | Cxcr | Foxa | Sox | | Nox |
| ments | 6 | 1 | 4 | 2 | 17 | Brachyury | 1 |

Spontaneously Differentiating EBs

| | | | | | | | |
|---|---|---|---|---|---|---|---|
| EB | — | — | — | — | — | — | — |
| AO1530 | NSC | NSC | ↑ | NCS | ↑ | NSC | NSC |
| tBHP | NSC | ↑ | NSC | ↓ | NSC | NSC | NSC |
| AO/tBHP | NSC | NSC | NSC | ↓ | ↓ | ↑ | NSC |

Activin A Treated EBs

| | | | | | | | |
|---|---|---|---|---|---|---|---|
| EB | — | — | — | — | — | — | — |
| AO1530 | NSC | NSC | NSC | NSC | NSC | NSC | ↑ |
| tBHP | NSC | NSC | NSC | NSC | NSC | NSC | ↑ |
| AO/tBHP | NSC | NSC | NSC | NSC | NSC | ↑ | ↑ |

BMP4 Treated EBs

| | | | | | | | |
|---|---|---|---|---|---|---|---|
| EB | — | — | — | — | — | — | — |
| AO1530 | NSC | NSC | NSC | NSC | NSC | NSC | NSC |
| tBHP | ↑↑↑ | NSC | NSC | NSC | NSC | ↑ | ↑ |
| AO/tBHP | ↑↑ | ↑ | NSC | NSC | ↓ | ↑↑ | ↑ |

RA Treated EBs

| | | | | | | | |
|---|---|---|---|---|---|---|---|
| EB | — | — | — | — | — | — | — |
| AO1530 | ↓ | ↓ | ↑ | NSC | NSC | ↓ | NSC |
| tBHP | NSC | NSC | ↑ | ↑ | ↑ | NSC | ↑ |
| AO/tBHP | ↓ | ↓ | ↑ | NSC | NSC | ↑ | ↑ |

TABLE 2

| Transcripts | Corrected | | | | [Control] | [Drug] | | | |
|---|---|---|---|---|---|---|---|---|---|
| Cluster Id | p-value | p-value | [Control](raw) | [Drug](raw) | (normalized) | (normalized) | Gene description | genbank | genesymbol |
| 6810592 | 0.028263254 | 1.36E−04 | 394.5062 | 187.68684 | 0.50325745 | −0.57134056 | fibroblast growth | BC048229 | Fgf10 |
| | | | | | | | factor 10 | | |
| 6848586 | 0.03890271 | 5.48E−04 | 8229.497 | 4804.9707 | 0.32084307 | −0.45950952 | brachyury | BC120807 | Bry |
| 6888114 | 0.028775461 | 1.54E−04 | 420.62076 | 806.02765 | −0.46519312 | 0.4739367 | neurogenic | BC018241 | Neurod1 |
| | | | | | | | differentiation 1 | | |
| 6952500 | 0.0208331 | 1.88E−05 | 3411.1348 | 2162.7986 | 0.32956442 | −0.32779375 | podocalyxin-like | BC054530 | Podxl |
| 6959468 | 0.030095968 | 2.06E−04 | 814.98 | 1029.3136 | −0.1732769 | 0.16368294 | mitogen-activated | BC005433 | Map4k1 |
| | | | | | | | protein kinase | | |
| | | | | | | | kinase kinase | | |
| | | | | | | | kinase 1 | | |
| 6962779 | 0.040422246 | 6.18E−04 | 2240.5337 | 1717.2704 | 0.17034023 | −0.21412499 | p21 (CDKN1A)- | AF082077 | Pak1 |
| | | | | | | | activated kinase 1 | | |
| 6965381 | 0.028775461 | 1.60E−04 | 2079.3152 | 3787.6875 | −0.48913416 | 0.37872887 | fibroblast growth | M30642 | Fgf4 |
| | | | | | | | factor 4 | | |
| 6992849 | 0.032521993 | 2.60E−04 | 2057.381 | 1071.638 | 0.44043383 | −0.5035699 | eomesodermin | BC094319 | Eomes |
| | | | | | | | homolog | | |
| | | | | | | | (Xenopus laevis) | | |

1. A method for differentiating an undifferentiated cell, said method comprising contacting an undifferentiated cell with a compound of Formula I or a salt thereof: 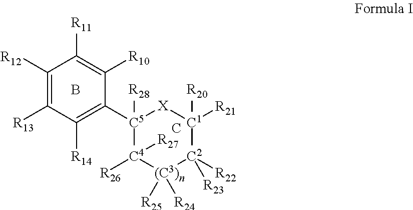
wherein:
A) R₁₂ and R₂₆ each independently represent —OH or a glycosidic functional group; R₁₀, R₁₁, R₁₃, and R₁₄ each independently represent H, —OH, nitro, halogen, amino, amido, cyano, carboxyl, sulphonyl, a glycosidic functional group, C₁₋₆ alkoxy-, hydroxy-C₁₋₆ alkyl-, C₁₋₆ alkoxy-C₁₋₆ alkyl-, or a saturated or unsaturated C₁₋₆ hydrocarbon chain which may be substituted with one or more of nitro, halogen, amino, amido, cyano, carboxyl, sulphonyl, hydroxyl, ketone or aldehyde groups; and wherein ring B comprises no more than one glycosidic functional group;
B) either a):
R₂₀ represents H or a C₂₋₃₀ saturated or unsaturated hydrocarbon chain;
R₂₁:
i) represents H;
ii) together with R₂₂ provides a second bond between C¹ and C²; or
iii) when X is —NR₁— and R is not H or C₁₋₆ alkyl, together with R₁ provides a second bond between C¹ and N;
R₂₂:
i) represents H;
ii) together with R₂₃ forms ═O; or
iii) together with R₂, provides a second bond between C¹ and C²;
R₂₃:
i) represents H or a C₂₋₃₀ saturated or unsaturated hydrocarbon chain; or
ii) together with R₂₂ forms ═O;
wherein at least one of R₂₀ and R₂₃ is a C₂₋₃₀ saturated or unsaturated hydrocarbon chain;
or b):
R₂₀, R₂₁, R₂₂, and R₂₃ form part of a 5, 6 or 7 membered unsaturated ring including C¹ and C², which ring is substituted with at least one group which is a C₂₋₃₀ saturated or unsaturated hydrocarbon chain, which ring is optionally and independently further substituted with one or more groups selected from nitro, halogen, amino, amido, cyano, carboxyl, sulphonyl, hydroxyl, ketone, aldehyde and saturated or unsaturated C₁₋₆ hydrocarbon chain, which C₁₋₆ hydrocarbon chain may be substituted with one or more of nitro, halogen, amino, amido, cyano, carboxyl, sulphonyl, hydroxyl, ketone, aldehyde or nitrone groups;
C) n is 0 or 1, wherein when n is 0, either i) R₂₇ and R₂₈ represent H or ii) R₂₇ together with R₂₈ provide a second bond between C⁴ and C⁵; or when n is 1, either i) R₂₄ and R₂₅ together form ═O and R₂₇ and R₂₈ represent H or R₂₇ together with R₂₈ provide a second bond between C⁴ and C⁵, or ii) R₂₄ and R₂₅ represent H and R₂₇ and R₂₈ represent H or R₂₇ together with R₂₈ provide a second bond between C⁴ and C⁵ or iii) R₂₄ represents H, R₂₅ together with R₂₇ provide a second bond between C³ and C⁴, R₂₈ represents —OH and X is —O—;
D) X is —O—, —S— or wherein R₁ i) represents H or C₁₋₆ alkyl, or ii) together with R₂, provides a second bond between C¹ and N;
wherein said C₂₋₃₀ saturated or unsaturated hydrocarbon chain of R₂₀, R₂₃ and the 5, 6 or 7 membered unsaturated ring is optionally and independently substituted with one or more groups selected from C₁₋₆ alkyl, C₁₋₆ alkoxy, hydroxy-C₁₋₆ alkyl, Cl, F, Br, I, —CN, —CO₂H, —CO₂C₁₋₆alkyl, —S(O)₂C₁₋₆alkyl, —S(O)₂-phenyl, —SC₁₋₆alkyl, —NO₂, —OH, —CF₃, —N(R₂)(R₃), —NHC(O)NHC₁₋₆alkyl, —C(O)N(R₂)(R₃), imine and substituted or unsubstituted triphenylphosphonium; and wherein one or more available —CH₂— groups present in the C₂₋₃₀ hydrocarbon chain of R₂₀, R₂₃ or the 5, 6 or 7 membered unsaturated ring is optionally and independently replaced by —O—, —C(O)—, —S(O)_{P}, or —N(R₂)—; wherein R₂ and R₃ each independently represent H or C₁₋₆ alkyl, and wherein p is 0 to 2;
and
wherein the total number of ═O on ring C is no greater than 1.

2. A method as claimed in claim 1, wherein X represents —O—.

3. A method as claimed in claim 1, wherein R₁₂ and R₂₆ both represent —OH.

4. A method as claimed in claim 1, wherein one of R₁₂ and R₂₆ represents —OH and the other of R₁₂ and R₂₆ represents a glycosidic functional group.

5. A method as claimed in claim 1, wherein n=1.

6. A method as claimed in claim 1, wherein n=0.

7. A method as claimed in claim 1, wherein:
R₂₀ represents H or a C₂₋₃₀ saturated or unsaturated hydrocarbon chain;
R₂₁:
i) represents H; or
ii) together with R₂₂ provides a second bond between C¹ and C²;
R₂₂:
i) represents H;
ii) together with R₂₃ forms ═O; or
iii) together with R₂, provides a second bond between C¹ and C²; and
R₂₃:
i) represents H or a C₂₋₃₀ saturated or unsaturated hydrocarbon chain; or
ii) together with R₂₂ forms ═O;

8. A method as claimed in claim 1, wherein R₂₀, R₂₁, R₂₂ and R₂₃, form part of a 5, 6 or 7 membered unsaturated ring including C¹ and C².

9. A method as claimed in claim 8, wherein said unsaturated ring is substituted with a C₂₋₃₀ saturated or unsaturated hydrocarbon chain at the meta or para position relative to C¹ or wherein said unsaturated ring is substituted with C₂₋₁₅ saturated or unsaturated hydrocarbon chains at two of the ortho, meta and para positions relative to C¹.

10. A method as claimed in claim 9, wherein said unsaturated ring is substituted with a C₂₋₃₀ saturated or unsaturated hydrocarbon chain at the meta position relative to C¹.

11. A method as claimed in claim 1, wherein the compound is a compound of Formula III or a salt thereof: 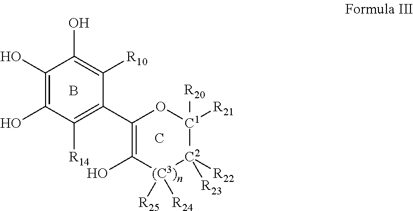
wherein:
A) R₁₀ and R₁₄ each represent H;
B) either a):
R₂₀ represents a C₂₋₁₄ saturated or unsaturated hydrocarbon chain; and
R₂₁ together with R₂₂ provides a second bond between C¹ and C²; and
R₂₃ represents H;
or b):
R₂₀, R₂₁, R₂₂ and R₂₃ form part of a 5, 6 or 7 membered unsaturated ring including C¹ and C² (“A” ring), which ring is substituted with at least one group which is a C₂₋₁₄ saturated or unsaturated hydrocarbon chain; and
C) R₂₄ and R₂₅ together form ═O.

12. A method as claimed in claim 11, wherein said C₂₋₃₀ saturated or unsaturated hydrocarbon chain of R₂₀, R₂₃ and the 5, 6 or 7 membered unsaturated ring includes a —CH₂— group connecting to C¹, C² or the 5, 6 or 7 membered ring.

13. A method as claimed in claim 12, wherein said C₂₋₃₀ saturated or unsaturated hydrocarbon chain of R₂₀, R₂₃ and the 5, 6 or 7 membered unsaturated ring is unsubstituted.

14. A method as claimed in claim 13, wherein said C₂₋₃₀ saturated or unsaturated hydrocarbon chain of R₂₀, R₂₃ and the 5, 6 or 7 membered unsaturated ring is saturated.

15. A method as claimed in claim 13 or claim 14, wherein said C₂₋₃₀ saturated or unsaturated hydrocarbon chain of R₂₀, R₂₃ and the 5, 6 or 7 membered unsaturated ring is a straight hydrocarbon chain comprising 6 to 15 carbon atoms.

16. A method as claimed in claim 11, wherein said 5, 6 or 7 membered unsaturated ring including C¹ and C² (“A” ring) is substituted with a C₂₋₃₀ saturated or unsaturated hydrocarbon chain at the meta position relative to C¹.

17. A method as claimed in claim 11, wherein the compound is selected from the group consisting of 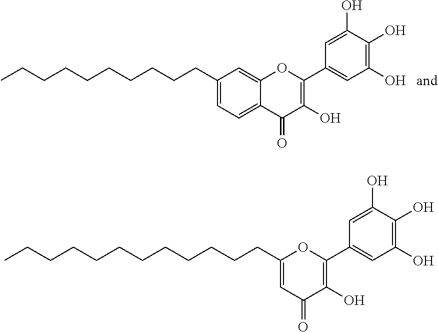
and salts of either thereof.

18. A method as claimed in claim 1, wherein the undifferentiated cell is a stem cell.

19. A method as claimed in claim 18, wherein the stem cell is a human embryonic stem cell.

20. (canceled)

21. A method as claimed in claim 18, wherein the stem cell is differentiated into a cell selected from the group consisting of epithelial cells, connective tissue cells, nerve cells, fat cells, pancreatic cells, liver cells, kidney cells, bone cells, hematopoietic cells, endothelial cells, retinal cells and smooth and striated muscle cells.

22-24. (canceled)
